(19) United States
(12) Patent Application Publication
(10) Pub. No.: US 2026/0272605 A1
(43) Pub. Date: Sep. 17, 2026

(54) Systems and Methods for Marking Skin

(71) Applicant: Viscot Medical LLC, East Hanover, NJ (US)

(72) Inventors: Gary J. PIERINGER, Bay Head, NJ (US); Gavin Joseph MCDONNELL, Morristown, NJ (US)

(21) Appl. No.: 19/566,627

(22) Filed: Mar. 13, 2026

Related U.S. Application Data
(60) Provisional application No. 63/771,471, filed on Mar. 13, 2025.

(51) Int. Cl. A61B 90/00

(52) U.S. Cl. CPC A61B 90/39; A61B 2090/395; A61B 2090/3966

(57) ABSTRACT

Methods and systems for marking skin are provided. A system for marking skin includes: a marker having a first end and a second end, the marker having a marking tip at the first end; a puck having at least a radiopaque portion; and a handle removably coupled to the second end of the marker. The methods and systems can reduce a user's (e.g., surgeon's) exposure to radiation from fluoroscopy during surgery. In some embodiments, the puck has a radiopaque edge that is visible with fluoroscopy. A handle, which may include a plurality of sections, may be used to position the puck. The handle is sufficiently long to allow the user to position the puck without exposing the user's hands to radiation during fluoroscopy. The radiopaque edge can aid the surgeon in identifying and/or marking a location for an incision.

### CROSS-REFERENCE TO RELATED APPLICATIONS

This application claims priority to U.S. Provisional Application 63/771,471 filed Mar. 13, 2025, the entire contents of which are incorporated herein by reference.

### FIELD

Embodiments of the present disclosure generally relate to systems and methods for marking skin, and, more particularly, for marking skin under fluoroscopy.

### BACKGROUND

Spinal disorders, such as herniated discs, degenerative disc disease, spinal stenosis, scoliosis, fractures, and tumors, can result from various causes, including trauma, degenerative conditions, and disease. These disorders often lead to significant symptoms, including pain, nerve compression, spinal instability, and loss of mobility, which may severely affect a patient's quality of life.

While non-surgical treatments such as physical therapy, medication, and lifestyle modifications may provide relief for some patients, many cases require surgical intervention to alleviate symptoms or prevent further deterioration. Common spinal surgical procedures include discectomy, laminectomy, spinal fusion, and vertebral fixation. These procedures frequently rely on imaging modalities, such as fluoroscopy, to guide surgical instruments and implants with precision.

Fluoroscopy is widely used in spinal surgery due to its ability to provide real-time imaging of the surgical site. However, fluoroscopy often entails surgeons using their hands or non-standard tools to indicate or mark points of interest under imaging, which may increase the surgeon's direct exposure to radiation. Furthermore, the use of non-standard tools for precise marking and fluoroscopy guidance can compromise the accuracy and efficiency of the procedure.

Therefore, the inventors propose methods and systems for marking skin using a dedicated tool that can facilitate precise imaging guidance, reduce surgeon exposure to fluoroscopic radiation, and improve surgical efficiency.

### SUMMARY

Methods and systems for marking skin are provided herein. In some embodiments, a system for marking skin includes: a marker having a first end and a second end, the marker having a marking tip at the first end; a puck having at least a radiopaque portion; and a handle removably coupled to the second end of the marker.

In some embodiments, a method for marking skin includes positioning a puck of a system for marking skin in a fluoroscopy field. The system includes: a marker having a first end and a second end, the marker having a marking tip at the first end; a puck having at least a radiopaque portion; and a handle removably coupled to the second end of the marker. The positioning is performed by a user manipulating the handle outside of the fluoroscopy field.

Other and further embodiments of the present disclosure are described below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 illustrates parts of a system for marking skin in an unassembled configuration in accordance with some embodiments of the present disclosure.

FIG. 2 illustrates the system shown in FIG. 1 in an assembled configuration.

FIG. 3 illustrates a side profile of a puck of the system shown in FIG. 1.

FIG. 4 illustrates a puck of the system shown in FIG. 1 in accordance with some embodiments of the present disclosure.

FIG. 5 illustrates a method of placing a portion of a system for marking skin in a fluoroscopy field in accordance with some embodiments of the present disclosure.

FIG. 6 illustrates a puck of the system shown in FIG. 4 remaining in a fluoroscopy field.

FIG. 7 illustrates a puck in an unassembled configuration in accordance with some embodiments of the present disclosure.

FIG. 8 illustrates an isometric top view of the puck shown in FIG. 7 in an assembled configuration.

FIG. 9 illustrates an isometric bottom view of the puck shown in FIG. 8.

FIG. 10 illustrate a right side view of the puck shown in FIG. 8.

FIG. 11 illustrates a rear view of the puck shown in FIG. 8.

FIG. 12 illustrates a front view of the puck shown in FIG. 8.

FIG. 13 illustrates a bottom view of the puck shown in FIG. 8.

FIG. 14 shows a puck placed on skin in accordance with some embodiments of the present disclosure.

FIG. 15 shows the puck of FIG. 14 in a fluoroscopy field with the puck in a first position.

FIG. 16 shows the puck of FIG. 14 moved to a second position.

FIG. 17 shows the puck of FIG. 16 without a fluoroscopy field.

FIG. 18 shows a mark on the skin.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Embodiments of methods and systems for marking skin under fluoroscopy are provided herein. More specifically, the present disclosure relates to a modular marker system specifically designed to enhance fluoroscopic guidance in spinal surgery and methods of use thereof. The systems and methods can advantageously reduce a user's exposure to radiation during fluoroscopic procedures.

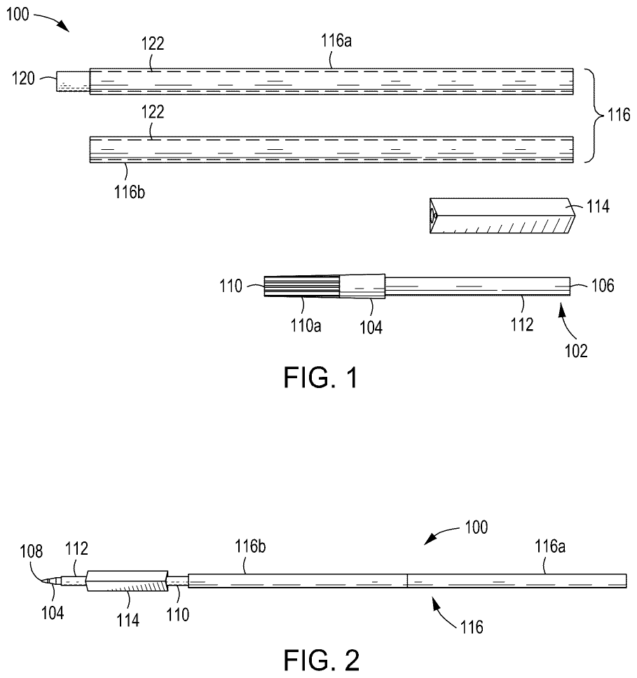

In embodiments, and as shown in FIGS. 1 and 2, a system 100 for marking skin includes a marker 102, a puck 114, and a handle 116. The marker 102 has a first end 104 and a second end 106. The marker has a marking tip 108 at the first end 104 to mark skin. The marking tip 108 may be covered, as shown in FIG. 1, with a removable cap 110. The marking tip 108 is configured for creating high-contrast marks on human tissue. In embodiments, the marker 102 is configured to make marks on skin that are durable and visible while the skin is in a fluoroscopic field and during surgery. In some embodiments, the marker 102 may include a supply of aseptic ink, such as gentian violet ink, coupled to the marking tip 108. In some embodiments, the marker 102 has a body 112, which may be hollow, and contains the aseptic ink. In some embodiments, an ink cartridge containing aseptic ink may be housed inside the body 112. The body 112 may be designed for easy handling and use with the other components of the system 100 described herein. The marker 102 may be made from at least one of radiolucent material, such as plastic, or radiopaque material, such as metal.

In some embodiments, the puck 114 of the system 100 may be configured as a shell that can be removably coupled to the marker 102 (as shown in FIG. 2). In such embodiments, the puck 114 (e.g., shell) is designed to fit securely over the body 112 of the marker 102. As described in greater detail herein, the puck 114 can be detached from the body 112 and left in place under fluoroscopy, serving as a visual reference point and guide for the surgeon.

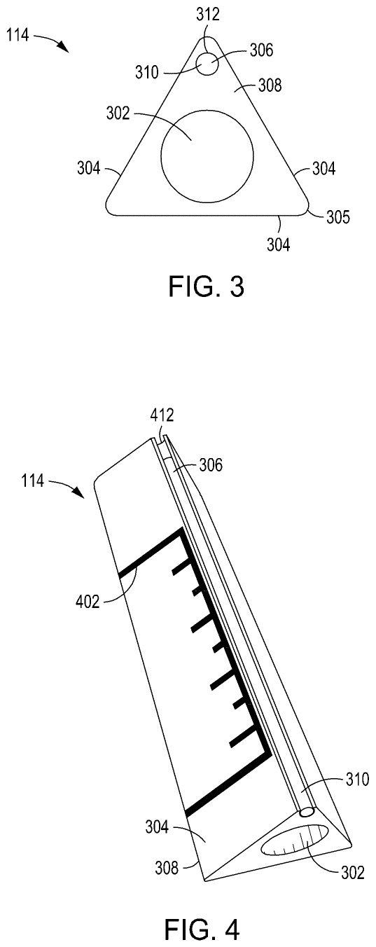

In embodiments, and as shown in FIG. 3, the puck 114 may have a longitudinal channel 302 configured to receive the marker 102. The channel 302 extends longitudinally through the puck 114 such that the marker 102 can be inserted into the channel 302 to retain the puck 114 on the marker 102. In some embodiments and as disclosed in FIG. 3, the channel 302 may be disposed completely within the puck 114. In some embodiments, the channel 302 may be only partially disposed within the puck 114 (e.g., formed in a side of the puck 114). In embodiments, and as shown in FIG. 2, the puck 114 may be removably coupled to the marker 102 at an intermediate location spaced between the first end 104 and the second end 106 of the marker 102.

In embodiments, and as shown in FIG. 3, the puck 114 has a profile having at least one flat side 304 (three flat sides 304 are shown in FIG. 3 creating a triangular profile, although other shapes including curved shapes having at least one flat side can be used). As described in greater detail below, the flat side(s) 304 may be useful to prevent the puck 114 from rolling or otherwise moving when placed during use.

In some embodiments, and as shown in FIG. 3, the puck 114 has a radiopaque portion 306, which enhances the puck's visibility under fluoroscopy. In some embodiments, the puck 114 may be made entirely of radiopaque material. In some embodiments, the puck 114 may be partially or completely coated with a radiopaque material (e.g., a metal foil or the like). The radiopaque portion 306 may be made of a metal, such as steel (e.g., stainless steel), nickel, or any other metal that has a high density. In some embodiments, the puck 114 includes a marking edge 305. In some embodiments, the marking edge is located proximate the radiopaque portion 306. For example, the marking edge 305 can be used to make a mark on a patient along the marking edge 305 after positioning the puck 114 under fluoroscopy with reference to the radiopaque portion 306. In some embodiments, the marking edge is linear, although other configurations of the marking edge can be used.

In some embodiments, and as shown in FIG. 3, the puck 114 may have a radiolucent portion 308. The radiolucent portion 308 may be made of plastic, rubber, radiolucent metals, or other radiolucent materials. In some embodiments, and as shown in FIG. 3, the radiopaque portion 306 may be disposed internally to the puck 114. As shown in FIG. 3, the radiopaque portion 306 may include an elongated metal rod 310 disposed in a longitudinal channel 312 of the radiolucent portion 308 of the puck 114. In some embodiments, the longitudinal channel 312 may extend along the entire length of the puck 114 or a portion thereof. In some embodiments, and as shown in FIG. 4, the radiopaque portion 306 may be disposed at least partially externally to the puck 114. As shown in FIG. 4, the metal rod 310 may be disposed in a longitudinal channel 412 formed in the side of the radiolucent portion 308. In embodiments, and as shown in FIGS. 3 and 4, the metal rod 310 is located on or near an edge of the puck 114 forming a radiopaque edge of the puck which is visible under fluoroscopy.

In some embodiments, the radiopaque portion 306 may include a plurality of radiopaque elements arranged in a linear pattern (e.g., along the length of the puck 114). The radiopaque elements may be spaced from one another in the linear pattern. For example, one or more pieces of metal may be fixed in the channel 412 shown in FIG. 4. The flat-sided geometry of the puck 114 and placement of the radiopaque portion facilitate clear imaging and precise localization during fluoroscopy and surgical procedure.

In some embodiments, the puck 114 may include indicia on one or more external surfaces of the puck 114, such as flat side 304 or a non-flat side. The indicia may include at least one of text, arrows, or measurement graduations (e.g., a ruler). For example, indicia 402 (measurement graduations) are shown on a flat side 304 of the puck 114 shown in FIG. 4. The indicia may be molded, engraved, etched, or otherwise formed in the surface of the puck 114 or may be applied externally onto the puck, such as by attaching a label, printing, or coating.

In embodiments, and as shown in FIGS. 1 and 2, the system 100 further includes handle 116 removably coupled to the second end 106 of the marker 102. At least a portion of the handle 116 is radiolucent and may be formed of plastic, rubber, radiolucent metals, or other radiolucent materials. In some embodiments, and as shown in FIGS. 1 and 2, the handle 116 may include one or more elongate rods or poles 116a, 116b that may be removably coupled together. The poles 116a, 116b may be modular to allow the poles 116a, 116b to connect together to extend the length of handle 116. In some embodiments, and as shown in FIG. 1, the poles 116a, 116b may be tubular and have a hollow central lumen 122 extending longitudinally. In some embodiments, the lumen 122 may not extend the entire length of the poles 116a, 116b, but may extend from one or both ends of the poles 116a, 116b as recesses. A coupler 120 may be inserted into the lumens 122 of opposing ends of the poles 116a, 116b to couple the poles 116a and 116b together. In some embodiments, if additional length of the handle 116 is desired, one or more additional couplers 120 may be used to modularly connect to additional poles 116a or 116b. In some embodiments, the handle 116 may extend to a total length of 1 to 4 feet. The extended configuration shown in FIG. 2 allows users (e.g., surgeons) to position the puck 114, in a fluoroscopy field without requiring direct proximity, thereby reducing radiation exposure to the user.

In some embodiments, the handle 116 may be connected indirectly to the second end 106 of the marker 102 through the cap 110, which may be used as a coupler between the handle 116 and the second end 106 of the marker 102. For example, in some embodiments, and as shown in FIG. 2, the cap 110 may be removed from the first end 104 of the marker 102 and attached to the second end 106 of the marker 102. At least a portion 110b of the cap 110 may be received in the lumen 122 of the pole 116b to thereby couple the pole 116b to the marker 102. Alternatively, the handle 116 may be configured to couple directly to the base of the marker 102 without using the cap 110 or using a coupler other than the cap 110.

In some embodiments, a user (e.g., a surgeon) may initially assemble the marker 102, the puck 114, and the handle 116 together as shown in FIG. 2. For example, the puck 114 may be slid over the body 112 securing the puck 114 to the marker 102. The second end 106 of the marker 102 may be coupled to the handle 116, forming an elongated assembly that allows the system 100 to be positioned precisely under fluoroscopy.

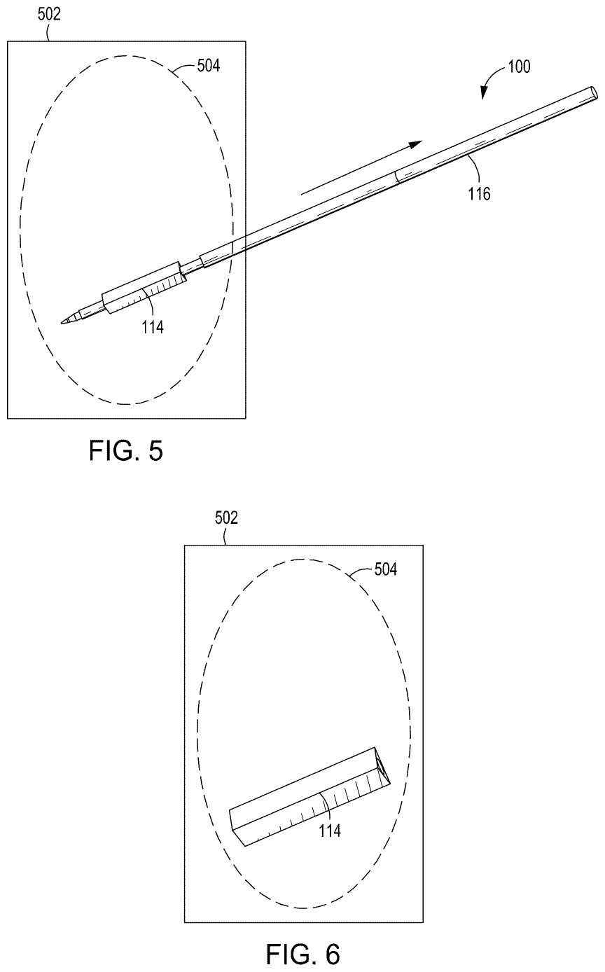

In embodiments, a method for marking skin may include positioning the puck 114 of the assembled system 100 for marking skin 502 in a fluoroscopy field 504 as shown, for example, in FIG. 5. The positioning may be performed by a user manipulating the handle 116 outside of the fluoroscopy field 504, which can limit a user's exposure to radiation. Positioning the puck 114 may include positioning the puck 114 so that the flat side 304 is positioned down towards the skin 502 of a patient. The method may include detaching the puck 114 from the marker 102 while the puck 114 remains in the fluoroscopy field 504, as shown, for example, in FIG. 6. For example, when a desired location of the puck 114 is identified while under fluoroscopy, the puck 114 may be detached from the body 112 so that the puck 114 remains under fluoroscopy as a stable, visible guide for the surgeon. This may eliminate the surgeon remaining in the fluoroscopy field 504, minimizing radiation exposure. In some embodiments, detaching the puck 114 from the marker 102 includes sliding the marker 102 through the longitudinal channel 302 as indicated by arrow in FIG. 5.

FIG. 6 illustrates a puck 714 in accordance with some embodiments of the present disclosure. Unless noted otherwise, the puck 714 shown in FIG. 6 may include any of the features of the puck 114 described above. In embodiments, the puck 714 may be designed to not be removably coupled to the marker 102. Instead, as described in greater detail herein, the puck 714 may be designed to be positioned on the skin of a patient using the handle 116 with or without the marker 102 by manipulating the puck 714 using the handle 116 without coupling the handle 116 and/or marker 102 to the puck 714.

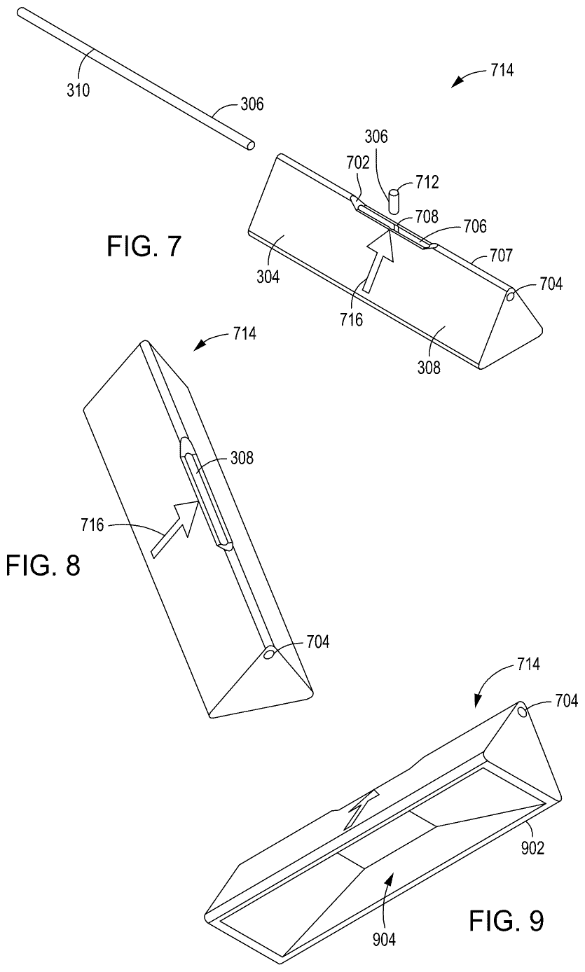

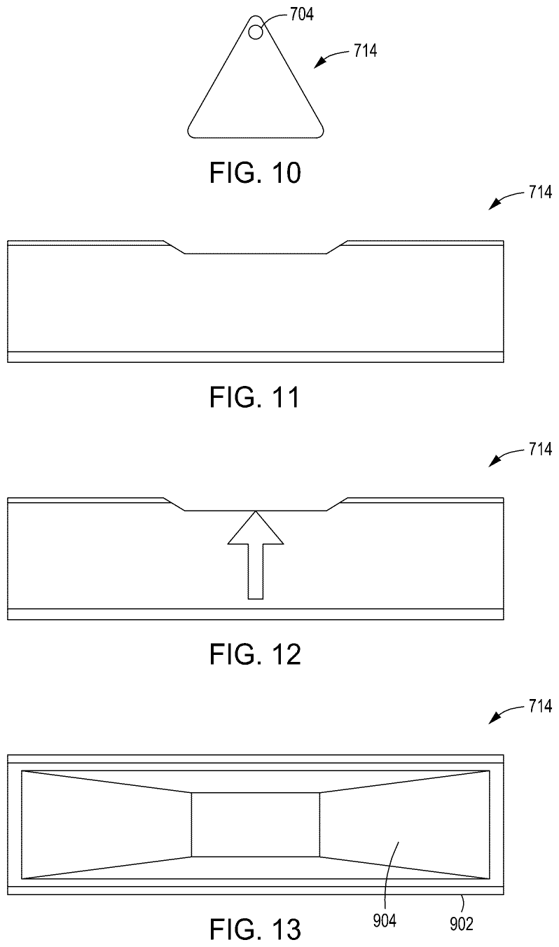

In some embodiments, and as shown in FIG. 7, the puck 714 has a profile having at least one flat side 304 (three flat sides 304 are shown in FIG. 7 creating a triangular profile, although other shapes including curved shapes having at least one flat side can be used). The flat side(s) 304 may be useful to prevent the puck 714 from rolling or otherwise moving when placed during use. The puck 714 may include the above-noted indicia on one or more external surfaces of the puck 714, such as flat side 304 or a non-flat side. In some embodiments, and as shown in FIG. 7, an arrow 716 is shown on one flat side 304. In some embodiments, the arrow 716 points toward a radiopaque portion 306 of the puck 714.

In some embodiments, and as shown in FIG. 7, the puck 714 has radiopaque portion 306 and a radiolucent portion 308. In some embodiments, and as shown in FIG. 8, the radiopaque portion 306 may be disposed at least partially internally to the puck 714. As shown in FIG. 7, the radiolucent portion 308 may include longitudinal channels 702 and 704 spaced apart by an opening 706, which may extend along an edge or corner 707 of the puck 714.

The longitudinal channels 702 and 704 and the opening are longitudinally aligned near or along the edge or corner 707 of the puck 714. In some embodiments, and as shown in FIG. 7, at least one of the longitudinal channels 702 or 704 may extend to a side of the puck 714 to permit insertion of a metal rod 310. In some embodiments and as shown in FIG. 7, the radiolucent portion 308 may also include a vertical channel 708 that intersects with the opening 706 at a non-zero angle (e.g., ninety degrees).

The radiopaque portion 306 may include the metal rod 310 and a metal pin 712. The metal rod 310 is configured to be disposed in at least one of the longitudinal channels 702 or 704 and extend at least partially across the opening 706 to at least cover the vertical channel 708. The metal pin 712 is configured to extend in the vertical channel 708. In embodiments, and as shown in FIG. 7, an alignment mark, such as the arrow 716 may align with the position of the metal pin 712.

During assembly of the puck 714, the metal pin 712 may be inserted into the vertical channel 708 so that metal pin 712 clears the opening 706. The metal rod 310 may be inserted through at least one of the longitudinal channels 702 or 704 so that the metal rod 310 extends at least over the metal pin 712, thereby retaining the metal pin 712 in the vertical channel 708. As shown in FIG. 8, the metal rod 310 extends at least partially in both longitudinal channels 702 and 704 and extends fully across the opening 706. In some embodiments, the metal rod 310 may extend the entire length of the puck 714.

The metal rod 310 and metal pin 712 form a pattern when viewed under fluoroscopy. When the puck 714 is viewed under fluoroscopy, the metal rod 310 appears as a radiopaque edge of the puck 714 and the metal pin 712 appears as a vertical alignment mark which may be used to align the puck 714 with a feature on the skin of the patient. The exposed portion of the metal rod 310 in the opening 706 can act as the marking edge for subsequent marking of a patient with the marker 102.

In some embodiments, and as shown in FIGS. 9 and 13, the puck 714 may have a recessed bottom 902 defining a cavity 904. The recessed cavity 904 reduces surface contact points to facilitate positioning of the puck 714 without tipping or wobbling. The recessed cavity 904 reduces friction with the skin to facilitate sliding of the puck 714 on the skin.

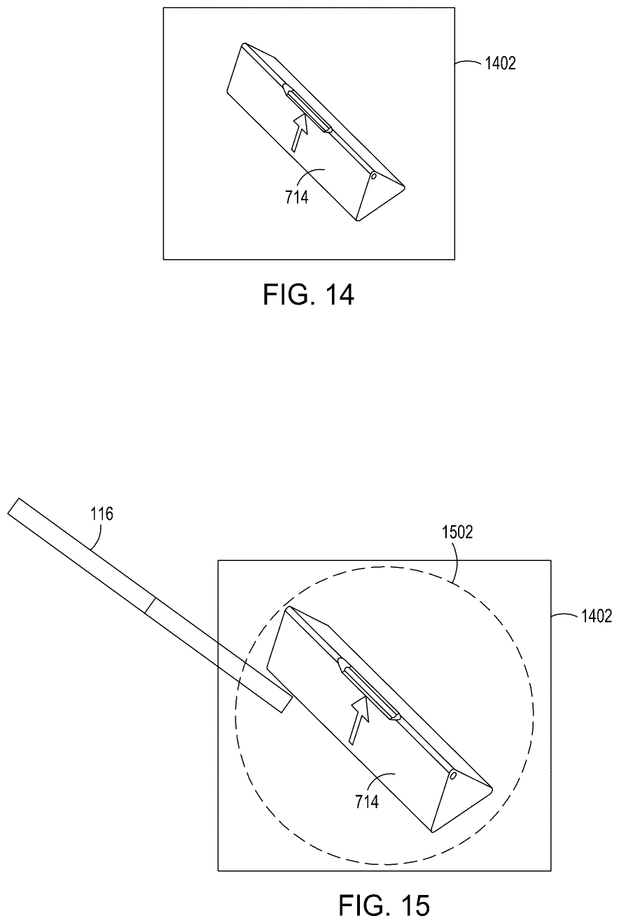

FIGS. 14 to 18 illustrate a method 1400 of using the system 100 according to some embodiments of the present disclosure. As shown in FIG. 14, the method 1400 may begin with a user (i.e., a surgeon) placing the puck 714 at a first position on the skin 1402 of a patient without the presence of a fluoroscopy field. Without the fluoroscopy field, the surgeon can directly handle the puck 714 and place it on the skin 1402.

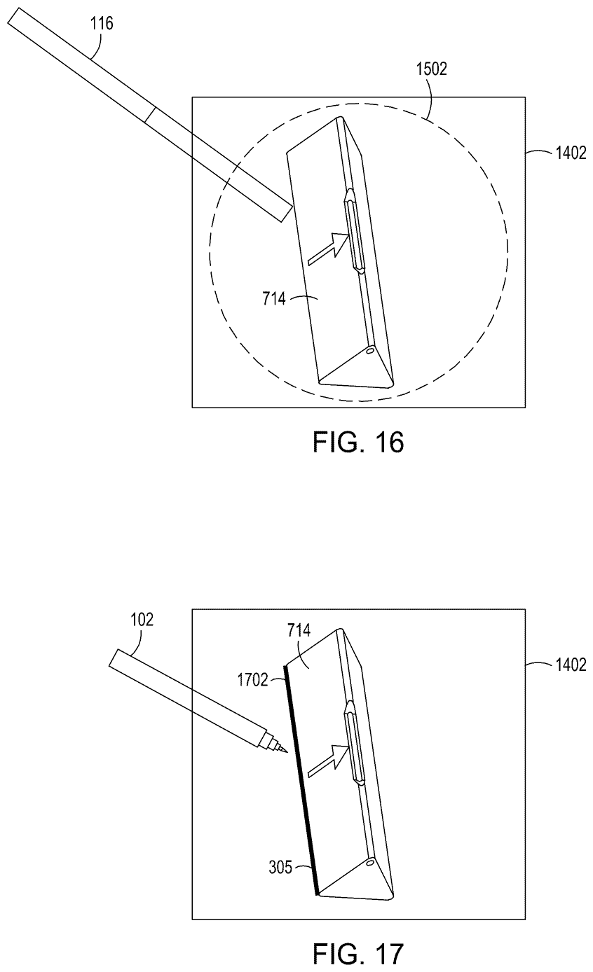

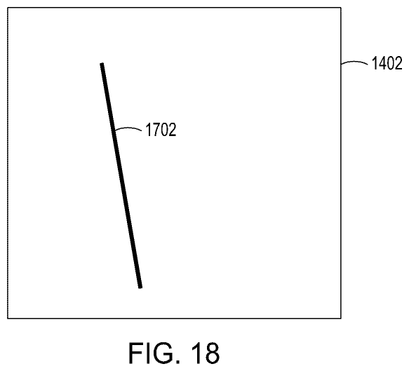

As shown in FIG. 15, the method 1400 may include turning on a fluoroscopy field 1502 around the puck 714 and inserting at least one of the handle 116 or the marker 102 into the fluoroscopy field 1502 to contact the puck 714 to move the puck 714 to a second position. FIG. 16 illustrates the puck 714 moved to the second position. As shown in FIG. 17, the method 1400 may include turning off the fluoroscopy field 1502 and using the marker 102 to make a mark 1702 (e.g., a line) on the skin 1402 along a portion (e.g., along the marking edge 305) of the puck 714. In some embodiments, turning off the fluoroscopy field 1502 can allow the surgeon to safely use one hand to hold the puck 714 (i.e., to prevent the puck 714 from moving relative to the skin 1402) and mark the skin 1402 with the marker 102 held in another hand. In some embodiments, the surgeon may leave the fluoroscopy field 1502 on and mark the skin 1402 using the marker 102 connected to the handle 116 so that the hands of the surgeon remain safely outside the fluoroscopy field 1502. As shown in FIG. 18, the method 1400 may include removing the puck 714 from the skin 1402, leaving the mark 1702 visible for further reference by the surgeon.

The modular design of the system 100 facilitates sterilization and storage of the individual components of the system 100, thus advantageously allowing reuse of the components rather than requiring the system to be disposable. The radiopaque portion 306 of the puck 114 and flat side(s) 304 provide superior fluoroscopic visibility and stability compared to existing tools. The elongated handle 116 enhances usability, enabling accurate placement of the puck 114 without the surgeon entering the fluoroscopy field, minimizing radiation exposure. The ability to position the puck 114 in a desired location under fluoroscopy and to leave the puck 114 in position for subsequent marking of the patient improves procedural efficiency by eliminating the need for repeated adjustments.

The systems and methods described herein may be used for surgical procedures where fluoroscopic imaging is used, such as in spinal surgeries requiring fluoroscopic imaging, including but not limited to procedures such as spinal fusions, laminectomies, discectomies, and vertebral fixation surgeries. The systems and methods provide a clear view of locations (e.g., spinal cord) to mark and/or make incisions for surgery. The systems and methods described herein offer a significant advancement in surgical instrumentation by addressing the limitations of current methods, reducing radiation exposure, and improving the precision and safety of spinal surgeries.

While the foregoing is directed to embodiments of the present disclosure, other and further embodiments of the disclosure may be devised without departing from the basic scope thereof.

1. A system for marking skin, comprising:
a marker having a first end and a second end, the marker having a marking tip at the first end;
a puck having at least a radiopaque portion; and
a handle removably coupled to the second end of the marker.

2. The system of claim 1, wherein the radiopaque portion is located proximate an edge of the puck.

3. The system of claim 1, wherein the marker includes a supply of aseptic ink coupled to the marking tip.

4. The system of claim 1, wherein the radiopaque portion includes a plurality of radiopaque elements arranged perpendicular to one another.

5. The system of claim 1, wherein at least a portion of at least one of the puck or the handle is radiolucent.

6. The system of claim 5, wherein the portion of at least one of the puck or the handle is formed of plastic.

7. The system of claim 1, wherein the puck has a profile having at least one flat side.

8. The system of claim 7, wherein the puck has a triangular profile.

9. The system of claim 1, wherein the handle includes a plurality of poles coupled together.

10. The system of claim 1, wherein the radiopaque portion includes an elongated metal rod.

11. The system of claim 1, wherein the radiopaque portion includes a plurality of radiopaque elements arranged in a linear pattern.

12. The system of claim 1, wherein the radiopaque portion is disposed internally to the puck.

13. The system of claim 1, wherein the radiopaque portion is disposed externally to the puck.

14. The system of claim 1, further comprising indicia on an external surface of the puck.

15. The system of claim 1, wherein the puck has a recessed bottom defining a cavity.

16. A method for marking skin, comprising:
positioning a puck of a system for marking skin in a fluoroscopy field, the system comprising:
a marker having a first end and a second end, the marker having a marking tip at the first end;
a puck having at least a radiopaque portion; and
a handle removably coupled to the second end of the marker,
wherein the positioning is performed by a user manipulating the handle outside of the fluoroscopy field.

17. The method of claim 16, further comprising marking the skin along a portion of the puck using the marker.

18. The method of claim 17, wherein the skin is marked along a linear marking edge of the puck.

19. The method of claim 16, wherein the puck has a profile having at least one flat side and wherein positioning the puck includes positioning the flat side down towards the skin.

20. The method of claim 16, further comprising:
turning off a fluoroscopy field after positioning the puck; and
marking the skin along a portion of the puck using the marker.
